(19) United States
(12) Patent Application Publication
(10) Pub. No.: US 2025/0378927 A1
(43) Pub. Date: Dec. 11, 2025

(54) DIGITAL IMAGING AND ARTIFICIAL INTELLIGENCE (AI)-BASED SYSTEMS AND METHODS FOR ANALYZING PRODUCT DOSING

(71) Applicant: The Procter & Gamble Company, Cincinnati, OH (US)

(72) Inventors: Sol Melissa ESCOBAR, Mason, OH (US); Kelly L. ANDERSON, Mason, OH (US); Meraj KHAN, Cincinnati, OH (US); Faiz Feisal SHERMAN, Mason, OH (US)

(21) Appl. No.: 19/228,830

(22) Filed: Jun. 5, 2025

Related U.S. Application Data
(60) Provisional application No. 63/656,765, filed on Jun. 6, 2024.

(51) Int. Cl. G16H 20/10; G06F 16/583; G06T 7/62; G06T 7/90; G06V 10/75

(52) U.S. Cl. CPC G16H 20/10; G06F 16/583; G06T 7/62; G06V 10/751; G06T 7/90; G06T 2207/20081

(57) ABSTRACT

Digital imaging and artificial intelligence (AI)-based systems and methods are described for analyzing pixel data of a product to determine product dosing. A product identifier of a product is detected, and a dosing application (app) receives a set of digital image(s) comprising pixel data depicting a dosage of the product and at least one of: (a) a product appliance configured to apply the product, or (b) a product implement configured to receive the product. An analysis is generated comprising a dosage comparison comparing the dosage of the product to a target dosage defining an expected dosage of the product at a first-time state. A feedback indication is output designed to address at least one feature identifiable within the pixel data comprising the dosage of the product.

### TECHNICAL FIELD

The present disclosure generally relates to digital imaging and artificial intelligence-based systems and methods, and, more particularly, to digital imaging and artificial intelligence (AI)-based systems and methods configured to analyze product dosing.

### BACKGROUND

Dosing compliance (such as the use of the appropriate recommended dose) in household products and common goods, as well as products that provide a clinical benefit, is a general behavioral barrier observed with consumers across various categories. For example, in the case of oral care, a category that in the United States is regulated by the Food and Drug Administration (FDA), adherence to proper product use is paramount not only for clinical benefit and efficacy reasons, but also for safety reasons. Oral care products can be considered as either medical drugs, medical devices, or even cosmetics. Most of these products contain specific active ingredients tested through clinical trials and monographs for both efficacy and safety. Studies have shown that a significant number of consumers underdose products such as fluoride toothpaste, teeth whitening treatments, and more. But the opposite can be true as well, where consumers can over-dose on such products. Because not all products have clear, legible usage instructions and/or because consumers do not read the instructions, it is difficult for consumers to know or keep track of their dosing across different products, such as oral care products. This is made even more difficult by products that require gradual increased or decreased dosing over time (such as to acclimate or build tolerance to the product), or a specific application method (e.g., such as with a denture adhesive on a removable denture).

For the foregoing reasons, there is a need for digital imaging and AI-based systems configured to analyze product dosing, which may include, for example, analyzing product usage in real-time or near real-time to provide feedback depicted as augmented reality (AR) based data overlayed or superimposed with a dosage of a product detected within one or more images (e.g., a video).

### SUMMARY

Generally, as described herein, digital imaging and AI-based systems are described for analyzing pixel data of digital images for determining or otherwise analyzing product dosing. Such digital imaging and AI-based systems provide a technical solution for overcoming problems that arise from the difficulties in identifying and using various products in a clinically effective manner and improving product efficacy for particular treatment applications for corresponding products.

The digital imaging and AI-based systems and methods as described herein allow a user to submit one or more images to imaging server(s) (e.g., including its one or more processors), or otherwise a computing device (e.g., such as locally on the user's mobile device), where the imaging server(s) or user computing device, implements or executes an AI-based learning model trained with pixel data of potentially 10,000s (or more) images depicting products and/or respective product dosages. The artificial intelligence model (e.g., a dosage learning model) may generate, based on pixel data of a given image, a feedback indication designed to address at least one feature identifiable within the pixel data comprising dosage of a given product (e.g., an oral product composition such as toothpaste). For example, an image of a product can comprise pixels or pixel data indicative of a dosage of a product, a product appliance (e.g., toothbrush) configured to apply the product, and/or a product implement (e.g., dentures) configured to receive the product. In some embodiments, the feedback indication may be transmitted via a computer network to a user computing device of the user for rendering on a display screen. In other embodiments, no transmission to the imaging server of the user's specific image occurs, where the feedback indication may instead be generated by the artificial intelligence model (e.g., a dosage learning model), executing and/or implemented locally on the user's mobile device and rendered, by a processor of the mobile device, on a display screen of the mobile device. In various embodiments, such rendering may include graphical representations, overlays, annotations, and the like for addressing the feature in the pixel data.

The digital imaging and AI-based systems and methods described herein reduces erroneous application or usage of a given product that is used, dosed, poured, dipped, or applied onto an implement or appliance, and provides immediate consumer feedback about whether and how much the product in question is under dosed, over dosed, or adequately dosed. In some aspects, a time factor can be relevant (as in the case of a gradual dose change product) and/or the pattern/shape of application can also be relevant (e.g., as in the case of denture adhesive), such that the digital imaging and AI-based systems and methods disclosed herein can also account for such changes, over time, and adapt the detection and feedback according to various time states.

The digital imaging and artificial intelligence-based systems and methods can provide various features or benefits over the existing art including, for example, the ability to instantaneously detect (e.g., in real-time or near real-time) one or more products, implements, and/or application from a digital library of identifiable products, implements, or appliances, and also to extract and provide data and information based on the interaction or otherwise relationships among the products, implements, or appliances (e.g., including formulation, specifications, or other attributes) and how those effect, correlate to, or otherwise apply to dosage efficacy and treatment. This also includes generating instantaneous or near instantaneous comparisons between those products, implements, and/or appliances based on different parameters (e.g., length, size, amount, chemical formulation, etc.) of such objects. This may also include adjustment of those comparisons based on a target state or otherwise dosage, which can be an expected dosage, and which can vary over time. Such implementation allows instantaneous output or otherwise feedback that allows the consumer to adjust his or her dosing usage or application, and, in various aspects, restart the process over again until the user meets the target dosage, which may be a clinical dosage or otherwise product efficacy-based dosage.

In some aspects, the techniques described herein relate to a digital imaging and artificial intelligence (AI)-based system configured to analyze product dosing, the digital imaging and AI-based system including: one or more processors; a dosing application (app) including computing instructions configured to execute on the one or more processors; and a dosing learning model, accessible by the dosing app, and trained with dosage data of one or more products, pixel data of a plurality of training images depicting the one or more products, and one or more product appliances or product implements associated with the one or more products, the dosing learning model trained to output analysis of one or more dosages corresponding to the one or more products based on one or more corresponding target dosages applied at different times during a product application lifecycle, wherein the computing instructions of the dosing app when executed by the one or more processors, cause the one or more processors to: detect, based on product data, a product identifier of a product, obtain a set of one or more images of the product, the set of one or more images including pixel data as captured by an imaging device, and the pixel data depicting a dosage of the product and at least one of: (a) a product appliance configured to apply the product, or (b) a product implement configured to receive the product, generate, based on output of the dosing learning model, a first analysis including a dosage comparison comparing the dosage of the product as depicted in pixel data to a target dosage of the product at a first time state, the target dosage of the product defining an expected dosage of the product at the first time state, and output, based on the dosage comparison, a feedback indication designed to address at least one feature identifiable within the pixel data including the dosage of the product.

In some aspects, the techniques described herein relate to a digital imaging and artificial intelligence (AI)-based method for analyzing product usage, the digital imaging and AI-based method including: detecting, by one or more processors based on product data, a product identifier of a product, obtaining, by a dosing application (app) executing on the one or more processors, a set of one or more images of the product, the set of one or more images including pixel data as captured by an imaging device, and the pixel data depicting a dosage of the product and at least one of: (a) a product appliance configured to apply the product, or (b) a product implement configured to receive the product, generating, based on output of a dosing learning model, a first analysis including a dosage comparison comparing the dosage of the product as depicted in pixel data to a target dosage of the product at a first time state, the target dosage of the product defining an expected dosage of the product at the first time state, wherein the dosing learning model executes on the one or more processors and is trained with dosage data of one or more products that includes the product, pixel data of a plurality of training images depicting the one or more products, and one or more product appliances or product implements associated with the one or more products, the dosing learning model trained to output analysis of one or more dosages corresponding to the one or more products based on one or more corresponding target dosages applied at different times during a product application lifecycle, and outputting, by the one or more processors based on the dosage comparison, a feedback indication designed to address at least one feature identifiable within the pixel data including the dosage of the product.

In some aspects, the techniques described herein relate to a tangible, non-transitory computer-readable medium storing instructions for analyzing product usage, that when executed by one or more processors cause the one or more processors to: detect, based on product data, a product identifier of a product, obtain, by a dosing application (app), a set of one or more images of the product, the set of one or more images including pixel data as captured by an imaging device, and the pixel data depicting a dosage of the product and at least one of: (a) a product appliance configured to apply the product, or (b) a product implement configured to receive the product, generate, based on output of a dosing learning model, a first analysis including a dosage comparison comparing the dosage of the product as depicted in pixel data to a target dosage of the product at a first time state, the target dosage of the product defining an expected dosage of the product at the first time state, wherein the dosing learning is trained with dosage data of one or more products that includes the product, pixel data of a plurality of training images depicting the one or more products, and one or more product appliances or product implements associated with the one or more products, the dosing learning model trained to output analysis of one or more dosages corresponding to the one or more products based on one or more corresponding target dosages applied at different times during a product application lifecycle, and output, based on the dosage comparison, a feedback indication designed to address at least one feature identifiable within the pixel data including the dosage of the product.

In accordance with the above, and with the disclosure herein, the present disclosure includes improvements in computer functionality or in improvements to other technologies at least because the disclosure describes that, e.g., an imaging server, or otherwise computing device (e.g., a user computer device), is improved where the intelligence or predictive ability of the server or computing device is enhanced by a trained (e.g., machine learning trained) dosing learning model. The dosing learning model, executing on the imaging server or computing device, is able to more accurately identify, based on pixel data of various products, feedback indications designed to address at least one feature identifiable within the pixel data comprising the dosage of the product. That is, the present disclosure describes improvements in the functioning of the computer itself or “any other technology or technical field” because an imaging server or user computing device is enhanced with a plurality of training images (e.g., 10,000s of training images and related pixel data as feature data) to accurately predict, detect, classify, or determine pixel data of a user-specific images, such as newly provided user images. This improves over the prior art at least because existing systems lack such predictive or classification functionality and are simply not capable of accurately analyzing user-specific images to output a predictive result to address at least one feature identifiable within the pixel data comprising the dosage of the product.

For similar reasons, the present disclosure relates to improvements to other technologies or technical fields at least because the present disclosure describes or introduces improvements to computing devices in the product dosing field, whereby the dosing learning model executing on the imaging device(s) or computing devices, improves the field of product dosing, chemical formulations and/or dosage identification and efficacy related thereto, with digital and/or artificial intelligence based analysis of product images to output a predictive result to address product related pixel data of at least one feature identifiable within the pixel data comprising the product, a product implement, a product application, a dosage of the product, and/or as otherwise describe herein.

In addition, the present disclosure relates to improvements to other technologies or technical fields at least because the present disclosure describes or introduces improvements to computing devices in the product dosage field, whereby the trained dosing learning model executing on the imaging device(s) or computing device(s) improve the underlying computer device (e.g., imaging server(s) and/or user computing device), where such computer devices are made more efficient by the configuration, adjustment, adaptation, and/or otherwise update of a given machine-learning network architecture. For example, in some embodiments, fewer machine resources (e.g., processing cycles or memory storage) may be used by decreasing computational resources by decreasing machine-learning network architecture needed to analyze images, including by reducing depth, width, image size, or other machine-learning based dimensionality requirements. Such reduction frees up the computational resources of an underlying computing system, thereby making it more efficient.

Still further, the present disclosure relates to improvement to other technologies or technical fields at least because the present disclosure describes or introduces improvements to computing devices in the field of security, where images of products are preprocessed (e.g., cropped or otherwise modified) to define extracted or depicted product portions of a product without depicting personal identifiable information (PII) of a user. For example, cropped or redacted portions of an image of a product may be used by the dosing learning model described herein, which eliminates the need of transmission of images that may include users using such products across a computer network (where such images may be susceptible of interception by third parties). Such features provide a security improvement, i.e., where the removal of PII (e.g., facial features) provides an improvement over prior systems because cropped or redacted images, especially ones that may be transmitted over a network (e.g., the Internet), are more secure without including PII information of a user. Accordingly, the systems and methods described herein operate without the need for such non-essential information, which provides an improvement, e.g., a security improvement, over prior systems. In addition, the use of cropped images, at least in some embodiments, allows the underlying system to store and/or process smaller data size images, which results in a performance increase to the underlying system as a whole because the smaller data size images require less storage memory and/or processing resources to store, process, and/or otherwise manipulate by the underlying computer system.

In addition, the present disclosure includes specific features other than what is well-understood, routine, conventional activity in the field, or adding unconventional steps that confine the claim to a particular useful application, e.g., digital imaging and AI-based systems and methods for analyzing product dosing, which may include, for example, analyzing product usage in real-time or near real-time to provide feedback depicted as AR based data overlayed or superimposed with a dosage of a product detected within one or more images (e.g., a video).

Advantages will become more apparent to those of ordinary skill in the art from the following description of the preferred embodiments which have been shown and described by way of illustration. As will be realized, the present embodiments may be capable of other and different embodiments, and their details are capable of modification in various respects. Accordingly, the drawings and description are to be regarded as illustrative in nature and not as restrictive.

### BRIEF DESCRIPTION OF THE DRAWINGS

The Figures described below depict various aspects of the system and methods disclosed therein. It should be understood that each Figure depicts an embodiment of a particular aspect of the disclosed system and methods, and that each of the Figures is intended to accord with a possible embodiment thereof. Further, wherever possible, the following description refers to the reference numerals included in the following Figures, in which features depicted in multiple Figures are designated with consistent reference numerals.

There are shown in the drawings arrangements which are presently discussed, it being understood, however, that the present embodiments are not limited to the precise arrangements and instrumentalities shown, wherein:

FIG. 1 illustrates an example digital imaging and AI-based system configured to analyze product dosing, in accordance with various embodiments disclosed herein.

FIG. 2A illustrates an example image and its related pixel data that may be used for training and/or implementing a product-based learning model, in accordance with various embodiments disclosed herein.

FIG. 2B illustrates an example image and its related pixel data that may be used for training and/or implementing a dosing learning model, in accordance with various embodiments disclosed herein.

FIG. 3 illustrates an example digital imaging and AI-based method for analyzing product dosing, in accordance with various embodiments disclosed herein.

FIG. 4 illustrates an example digital imaging and AI-based method for analyzing product dosing including analyzing product dosing across a plurality of time states, in accordance with various embodiments disclosed herein.

FIG. 5 illustrates an example user interface as rendered on a display screen of a user computing device in accordance with various embodiments disclosed herein.

The Figures depict preferred embodiments for purposes of illustration only. Alternative embodiments of the systems and methods illustrated herein may be employed without departing from the principles of the invention described herein.

### DETAILED DESCRIPTION OF THE INVENTION

To define more clearly the terms used herein, the following definitions are provided. Unless otherwise indicated, the following definitions are applicable to this disclosure. If a term is used in this disclosure but is not specifically defined herein, the definition from the IUPAC Compendium of Chemical Terminology, 2nd Ed (1997), can be applied, as long as that definition does not conflict with any other disclosure or definition applied herein, or render indefinite or non-enabled any claim to which that definition is applied.

The term “oral product composition”, as used herein, includes a product, which in the ordinary course of usage, is not intentionally swallowed for purposes of systemic administration of particular therapeutic agents, but is rather retained in the oral cavity for a time sufficient to contact dental surfaces or oral tissues. Examples of oral product compositions include dentifrice, toothpaste, tooth gel, subgingival gel, emulsion, mouth rinse, mousse, foam, mouth spray, lozenge, chewable tablet, chewing gum, tooth whitening strips, floss and floss coatings, breath freshening dissolvable strips, unit-dose composition, fibrous composition, or denture care or adhesive product. The oral product composition may also be incorporated onto strips or films for direct application or attachment to oral surfaces, such as tooth whitening strips. Examples of emulsion compositions include the emulsions compositions of U.S. Pat. No. 11,147,753, jammed emulsions, such as the jammed oil-in-water emulsions of U.S. Pat. No. 11,096,874. Examples of unit-dose compositions include the unit-dose compositions of U.S. Patent Application Publication No. 2019/0343732.

The term “dentifrice composition”, as used herein, includes tooth or subgingival-paste, gel, or liquid formulations unless otherwise specified. The dentifrice composition may be a single-phase composition or may be a combination of two or more separate dentifrice compositions. The dentifrice composition may be in any desired form, such as deep striped, surface striped, multilayered, having a gel surrounding a paste, or any combination thereof. Each dentifrice composition in a dentifrice comprising two or more separate dentifrice compositions may be contained in a physically separated compartment of a dispenser and dispensed side-by-side.

“Active and other ingredients” useful herein may be categorized or described herein by their cosmetic and/or therapeutic benefit or their postulated mode of action or function. However, it is to be understood that the active and other ingredients useful herein can, in some instances, provide more than one cosmetic and/or therapeutic benefit or function or operate via more than one mode of action. Therefore, classifications herein are made for the sake of convenience and are not intended to limit an ingredient to the particularly stated function(s) or activities listed.

The term “substantially free” as used herein refers to the presence of no more than 0.05%, preferably no more than 0.01%, and more preferably no more than 0.001%, of an indicated material in a composition, by total weight of such composition.

The term “essentially free” as used herein means that the indicated material is not deliberately added to the composition, or preferably not present at analytically detectable levels. It is meant to include compositions whereby the indicated material is present only as an impurity of one of the other materials deliberately added.

The term “oral hygiene regimen” or “regimen” can be for the use of two or more separate and distinct treatment steps for oral health, e.g., toothpaste, mouth rinse, floss, toothpicks, spray, water irrigator, massager.

While compositions and methods are described herein in terms of “comprising” various components or steps, the compositions and methods can also “consist essentially of” or “consist of” the various components or steps, unless stated otherwise.

As used herein, the word “or” when used as a connector of two or more elements is meant to include the elements individually and in combination; for example, X or Y, means X or Y or both.

As used herein, the articles “a” and “an” are understood to mean one or more of the material that is claimed or described, for example, the singular “an oral product composition” or “a bleaching agent” may also include the plural unless the context specifically states otherwise.

Several types of ranges are disclosed in relation to embodiments of the present invention. When a range of any type is disclosed or claimed, the intent is to disclose or claim individually each possible number that such a range could reasonably encompass, including end points of the range as well as any sub-ranges and combinations of sub-ranges encompassed therein.

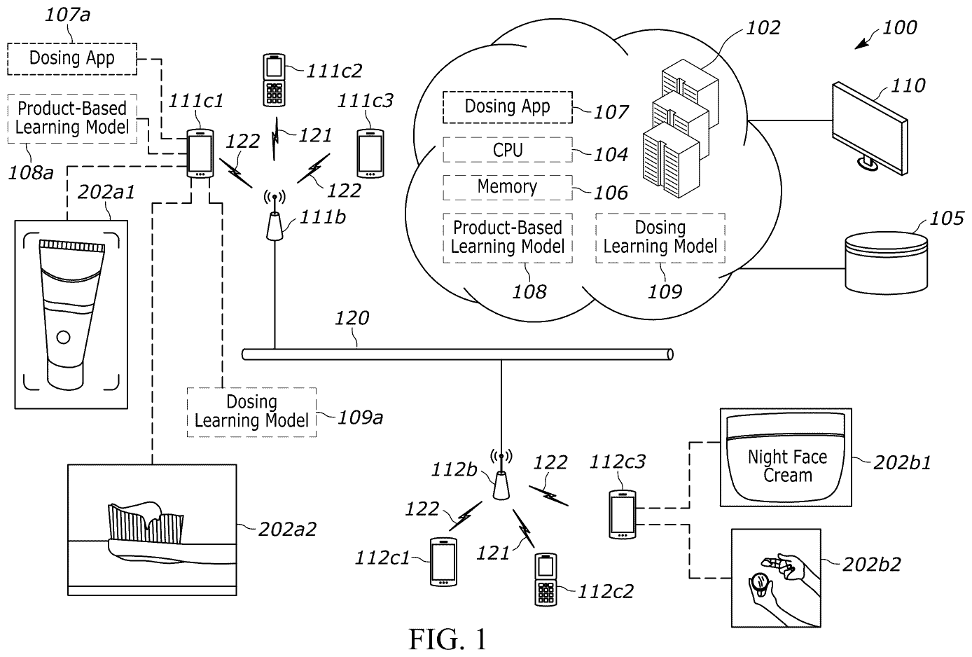

FIG. 1 illustrates an example digital imaging and artificial intelligence (AI)-based system 100 configured to analyze product dosing, in accordance with various embodiments disclosed herein. In the example embodiment of FIG. 1, digital imaging and AI-based system 100 includes server 102, which may comprise one or more computer servers. In various embodiments, server 102 may comprise multiple servers, which may comprise multiple, redundant, or replicated servers as part of a server farm. In still further embodiments, server 102 may be implemented as cloud-based server(s), such as a cloud-based computing platform. For example, imaging server 102 may be any one or more cloud-based platform(s) such as MICROSOFT AZURE, AMAZON AWS, or the like. Server 102 may include one or more processor 104 (i.e., CPU(s)) as well as one or more computer memories 106. In various embodiments, server 102 may be referred to herein as “imaging server(s).” Memory 106 may include one or more forms of volatile and/or non-volatile, fixed and/or removable memory, such as read-only memory (ROM), electronic programmable read-only memory (EPROM), random access memory (RAM), erasable electronic programmable read-only memory (EEPROM), and/or other hard drives, flash memory, MicroSD cards, and others.

Memory 106 may store an operating system (OS) (e.g., Microsoft Windows, Linux, UNIX, etc.) capable of facilitating the functionalities, apps, methods, or other software as discussed herein. Memory 106 may store a product-based learning model 108, which may comprise an artificial intelligence-based model, such as a machine learning model, trained on various images (e.g., images 202a1 and/or 202b1), as described herein. Memory 106 may also store a dosing learning model 109, which may comprise an artificial intelligence-based model, such as a machine learning model, trained on various images (e.g., images 202a2 and/or 202b2), as described herein. Additionally, or alternatively, product-based learning model 108 and/or dosing learning model 109 may also be stored in database 105, which is accessible or otherwise communicatively coupled to imaging server 102. In addition, memories 106 may also store machine readable instructions, including any of one or more application(s) (e.g., an dosing application as described herein), one or more software component(s), and/or one or more application programming interfaces (APIs), which may be implemented to facilitate or perform the features, functions, or other disclosure described herein, such as any methods, processes, elements or limitations, as illustrated, depicted, or described for the various flowcharts, illustrations, diagrams, figures, and/or other disclosure herein. For example, at least some of the applications, software components, or APIs may be, include, otherwise be part of, a machine learning model or component, such as the product-based learning model 108, product-based learning model 108a, dosing learning model 109, and/or product-based learning model 109a, where each may be configured to facilitate their various functionalities discussed herein. It should be appreciated that one or more other applications may be envisioned and that are executed by the processor 104.

Processor 104 may be connected to the memories 106 via a computer bus responsible for transmitting electronic data, data packets, or otherwise electronic signals to and from processor 104 and memories 106 in order to implement or perform the machine-readable instructions, methods, processes, elements or limitations, as illustrated, depicted, or described for the various flowcharts, illustrations, diagrams, figures, and/or other disclosure herein.

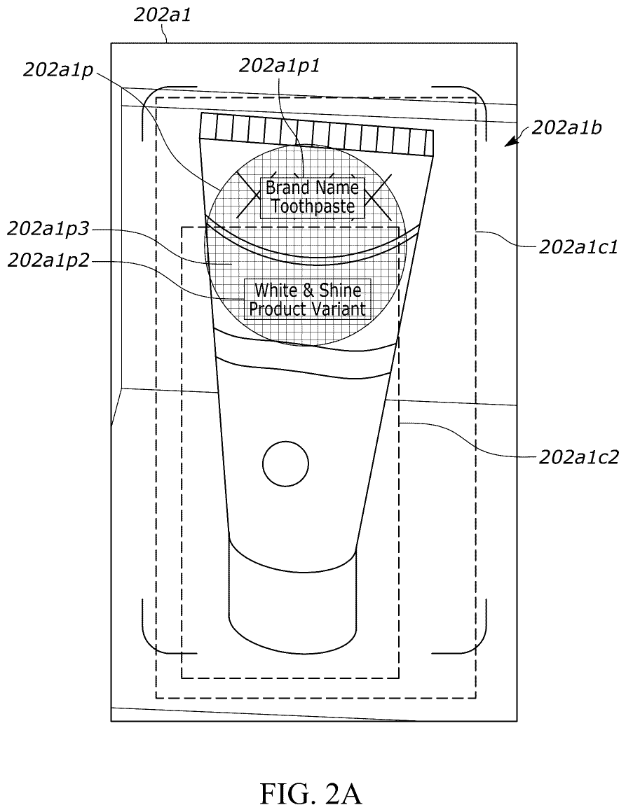

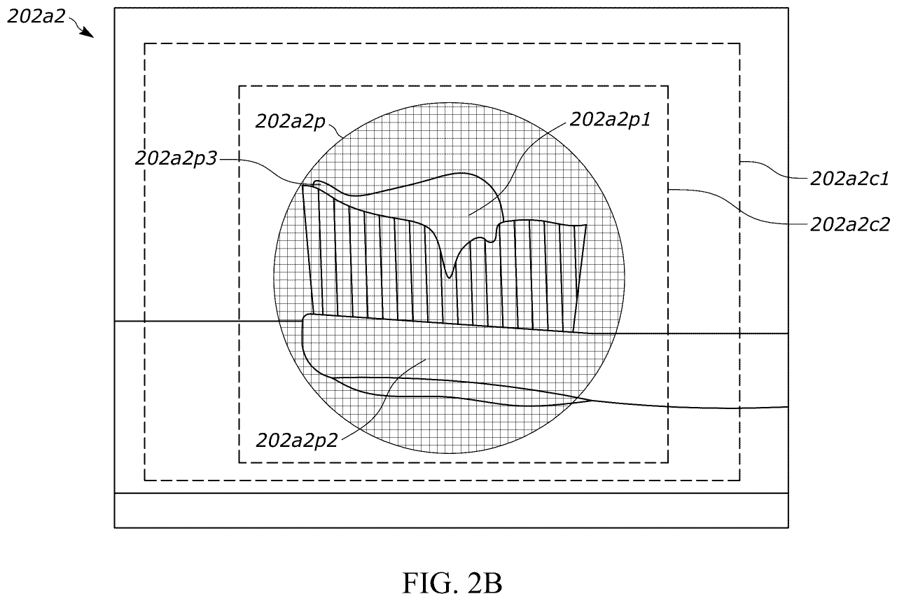

Processor 104 may interface with memory 106 via the computer bus to execute an operating system (OS). Processor 104 may also interface with the memory 106 via the computer bus to create, read, update, delete, or otherwise access or interact with the data stored in memories 106 and/or the database 105 (e.g., a relational database, such as Oracle, DB2, MySQL, or a NoSQL based database, such as MongoDB). The data stored in memories 106 and/or database 105 may include all or part of any of the data or information described herein, including, for example, training images and/or user images (e.g., including any one or more of images 202a1, 202a2, 202b1, 202b2, and/or zoomed, cropped, and/or segmentation related images for example as shown for FIGS. 2A and/or 2B), and/or other images and/or information of products, or other information or data as otherwise described herein.

Imaging server 102 may further include a communication component configured to communicate (e.g., send and receive) data via one or more external/network port(s) to one or more networks or local terminals, such as computer network 120 and/or terminal 110 (for rendering or visualizing) described herein. In some embodiments, imaging server 102 may include a client-server platform technology such as ASP.NET, Java J2EE, Ruby on Rails, Node.js, a web service or online API, responsive for receiving and responding to electronic requests. The imaging server 102 may implement the client-server platform technology that may interact, via the computer bus, with the memories 106 (including the application(s), component(s), API(s), data, etc. stored therein) and/or database 105 to implement or perform the machine readable instructions, methods, processes, elements or limitations, as illustrated, depicted, or described for the various flowcharts, illustrations, diagrams, figures, and/or other disclosure herein.

In various embodiments, imaging server 102 may include or interact with one or more transceivers (e.g., WWAN, WLAN, and/or WPAN transceivers) functioning in accordance with IEEE standards, 3GPP standards, or other standards, and that may be used in receipt and transmission of data via external/network ports connected to computer network 120. In some embodiments, computer network 120 may comprise a private network or local area network (LAN). Additionally, or alternatively, computer network 120 may comprise a public network such as the Internet.

Imaging server 102 may further include or implement an operator interface configured to present information to an administrator or operator and/or receive inputs from the administrator or operator. As shown in FIG. 1, an operator interface may provide a display screen (e.g., via terminal 110). Imaging server 102 may also provide I/O components (e.g., ports, capacitive or resistive touch sensitive input panels, keys, buttons, lights, LEDs), which may be directly accessible via, or attached to, imaging server 102 or may be indirectly accessible via or attached to terminal 110. According to some embodiments, an administrator or operator may access the server 102 via terminal 110 to review information, make changes, input training data or images, initiate training of dosing learning model 109, and/or perform other functions.

As described herein, in some embodiments, imaging server 102 may perform the functionalities as discussed herein as part of a “cloud” network or may otherwise communicate with other hardware or software components within the cloud to send, retrieve, or otherwise analyze data or information described herein.

In general, a computer program or computer based product, application, or code (e.g., the model(s), such as AI models, or other computing instructions described herein) may be stored on a computer usable storage medium, or tangible, non-transitory computer-readable medium (e.g., standard random access memory (RAM), an optical disc, a universal serial bus (USB) drive, or the like) having such computer-readable program code or computer instructions embodied therein, wherein the computer-readable program code or computer instructions may be installed on or otherwise adapted to be executed by the processor 104 (e.g., working in connection with the respective operating system in memories 106) to facilitate, implement, or perform the machine readable instructions, methods, processes, elements or limitations, as illustrated, depicted, or described for the various flowcharts, illustrations, diagrams, figures, and/or other disclosure herein. In this regard, the program code may be implemented in any desired program language, and may be implemented as machine code, assembly code, byte code, interpretable source code or the like (e.g., via Golang, Python, C, C++, C#, Objective-C, Java, Scala, ActionScript, JavaScript, HTML, CSS, XML, etc.).

As shown in FIG. 1, imaging servers 102 are communicatively connected, via computer network 120 to the one or more user computing devices 111c-111c3 and/or 112c1-112c3 via base stations 111b and 112b. In some embodiments, base stations 111b and 112b may comprise cellular base stations, such as cell towers, communicating to the one or more user computing devices 111c1-111c3 and 112c1-112c3 via wireless communications 121 based on any one or more of various mobile phone standards, including NMT, GSM, CDMA, UMMTS, LTE, 5G, or the like. Additionally, or alternatively, base stations 111b and 112b may comprise routers, wireless switches, or other such wireless connection points communicating to the one or more user computing devices 111c1-111c3 and 112c1-112c3 via wireless communications 122 based on any one or more of various wireless standards, including by non-limiting example, IEEE 802.11a/b/c/g (WIFI), the BLUETOOTH standard, or the like.

Any of the one or more user computing devices 111c1-111c3 and/or 112c1-112c3 may comprise mobile devices and/or client devices for accessing and/or communications with imaging server 102. Such mobile devices may comprise one or more mobile processor(s) and/or an imaging device for capturing images, such as images as described herein (e.g., any one or more of images 202a1, 202a2, 202b1, and/or 202b2). In various embodiments, user computing devices 111c1-111c3 and/or 112c1-112c3 may comprise a mobile phone (e.g., a cellular phone), a tablet device, a personal data assistance (PDA), or the like, including, by non-limiting example, an APPLE IPHONE or IPAD device or a GOOGLE ANDROID based mobile phone or table.

In various embodiments, the one or more user computing devices 111c1-111c3 and/or 112c1-112c3 may implement or execute an operating system (OS) or mobile platform such as Apple's iOS and/or Google's ANDROID operation system. Any of the one or more user computing devices 111c1-111c3 and/or 112c1-112c3 may comprise one or more processors and/or one or more memories for storing, implementing, or executing computing instructions or code, e.g., a mobile application or a home or personal assistant application, as described in various embodiments herein. As shown in FIG. 1, product-based learning model 108a, dosing app 107a, and/or dosing learning model 109a as described herein, or at least portions thereof, may also be stored locally on a memory of a user computing device (e.g., user computing device 111c1). In some aspects, product-based learning model 108a, dosing app 107a, and/or dosing learning model 109a as installed on a computing device may comprise a same product-based learning model, dosing app, and/or dosing learning model as installed on server 102. Additionally, or alternatively, product-based learning model 108a, dosing app 107a, and/or dosing learning model 109a may comprise a portion of the product-based learning model 108, dosing app 107, and/or dosing learning model 109 as installed on server 102, where such respective models can communicate with each other across computer network 120. Further, it is to be understood that in some aspects, product-based learning model, doing app, and product-based learning model may be installed wholly at user computing device, wholly at server 102, or partially on user computing device and partially on server 102 where communication between dosing learning model 109a and dosing learning model 109, between dosing app 107a and dosing app 107, and product-based learning model 108 and product-based learning model 108a, occurs through computer network 120. Generally, when a given model or app is referred to herein, it refers respectively to one or both of the given app or model, whether operating alone at the sever or computing device, or whether communicating over computer network 120.

User computing devices 111c1-111c3 and/or 112c1-112c3 may comprise a wireless transceiver to receive and transmit wireless communications 121 and/or 122 to and from base stations 111b and/or 112b. In various embodiments, pixel-based images (e.g., images 202a1, 202a2, 202b1, and/or 202b2) may be transmitted via computer network 120 to imaging server 102 for training of model(s) (e.g., dosing learning model and/or product-based learning model) and/or imaging analysis as described herein.

In addition, the one or more user computing devices 111c1-111c3 and/or 112c1-112c3 may include an imaging device (e.g., a camera) and/or digital video camera for capturing or taking digital images and/or frames (e.g., which can be any one or more of images 202a1, 202a2, 202b1, and/or 202b2). Each digital image may comprise pixel data for training or implementing model(s), such as AI or machine learning models, as described herein. For example, an imaging device and/or digital video camera of, e.g., any of user computing devices 111c1-111c3 and/or 112c1-112c3, may be configured to take, capture, or otherwise generate digital images (e.g., pixel-based images 202a1, 202a2, 202b1, and/or 202b2) and, at least in some embodiments, may store such images in a memory of a respective user computing device. Additionally, or alternatively, such digital images may also be transmitted to and/or stored on memory 106 and/or database 105 of server 102.

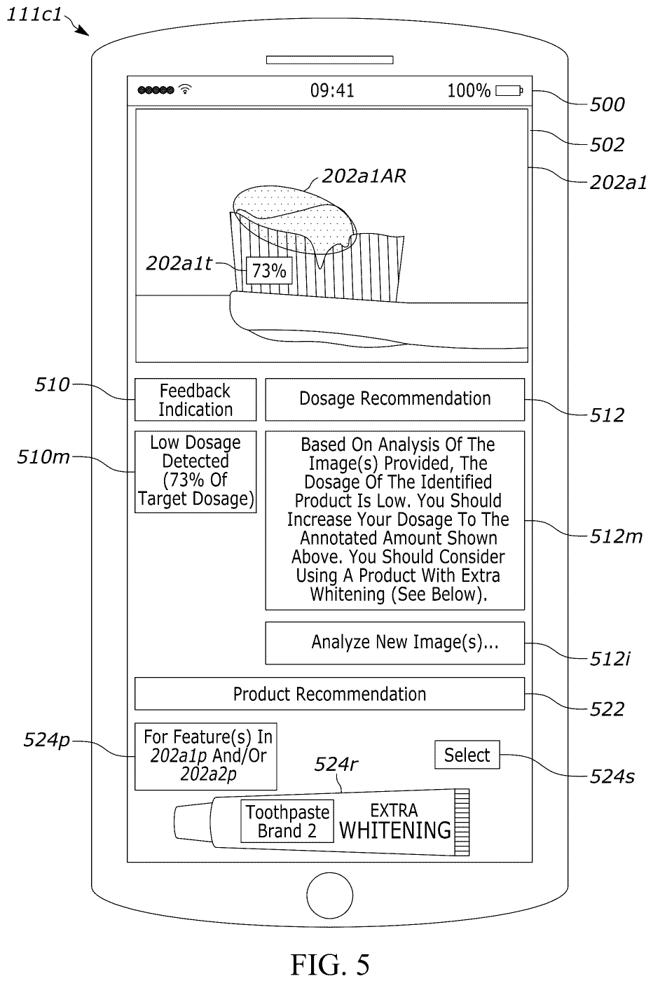

Still further, each of the one or more user computer devices 111c1-111c3 and/or 112c1-112c3 may include a display screen for displaying graphics, images, text, product(s), data, pixels, features, and/or other such visualizations or information as described herein. In various embodiments, graphics, images, text, product(s), data, pixels, features, and/or other such visualizations or information may be received from imaging server 102 for display on the display screen of any one or more of user computer devices 111c1-111c3 and/or 112c1-112c3. Additionally, or alternatively, a user computer device, e.g., as described herein for FIG. 5, may comprise, implement, have access to, render, or otherwise expose, at least in part, an interface or a guided user interface (GUI) for displaying text and/or images on its display screen.

In some embodiments, computing instructions and/or applications executing at the server (e.g., server 102) and/or at a mobile device (e.g., mobile device 111c1) may be communicatively connected for analyzing pixel data of an image of a product to generate or otherwise output a feedback indication(s) designed to address features identifiable within the pixel data comprising the dosage of the product, as described herein. For example, one or more processors (e.g., processor 104) of server 102 may be communicatively coupled to a mobile device via a computer network (e.g., computer network 120). In such embodiments, a dosing app may comprise a server app portion configured to execute on the one or more processors of the server (e.g., server 102) and a mobile app portion configured to execute on one or more processors of the mobile device (e.g., any of one or more user computing devices 111c1-111c3 and/or 112c1-112c3). In such embodiments, the server app portion is configured to communicate with the mobile app portion. The server app portion or the mobile app portion may each be configured to implement, or partially implement, one or more of: (1) detecting, based on the product data, the product identifier of the product; (2) obtaining the set of one or more images of the product; (3) generating, based on the output of the dosing learning model, the first analysis comprising a dosage comparison; and/or (4) outputting, based on the dosage comparison, the feedback indication.

FIG. 2A illustrates an example image 202a1 and its related pixel data that may be used for training and/or implementing a product-based learning model, in accordance with various embodiments disclosed herein. In various embodiments, as shown for FIG. 2A, image 202a1 may be an image captured by a user. More generally, image 202a1 (as well as images 202a2, 202b1 and/or 202b2) may be transmitted to server 102 via computer network 120, as shown for FIG. 1. It is to be understood that such images may be captured by the users themselves or, additionally or alternatively, others, where such images are used and/or transmitted on behalf of a user.

Digital images, such as non-limiting example images 202a1, 202a2, 202b1 and/or 202b2, may be collected or aggregated at imaging server 102 and may be analyzed by, and/or used to train, an AI-based model (e.g., an AI model such as a machine learning imaging model as described herein). Each of these images may comprise pixel data comprising feature data and corresponding to product(s), product dosage(s), product implement(s), product appliance(s), background(s), and/or other features described herein. The pixel data may be captured by an imaging device of one of the user computing devices (e.g., one or more user computer devices 111c1-111c3 and/or 112c1-112c3).

With respect to digital images as described herein, pixel data (e.g., pixel data 202a1p of FIG. 2A) comprises individual points or squares of data within an image, where each point or square represents a single pixel (e.g., each of pixel 202a1p1, pixel 202a1p2, and pixel 202a1p3) within an image. Each pixel may be at a specific location within an image. In addition, each pixel may have a specific color (or lack thereof). Pixel color may be determined by a color format and related channel data associated with a given pixel. For example, a popular color format is a 1976 CIELAB (also referenced herein as the “CIE L*-a*-b*” or simply “L*a*b*” color format) color format that is configured to mimic the human perception of color. Namely, the L*a*b* color format is designed such that the amount of numerical change in the three values representing the L*a*b* color format (e.g., L*, a*, and b*) corresponds roughly to the same amount of visually perceived change by a human. This color format is advantageous, for example, because the L*a*b* gamut (e.g., the complete subset of colors included as part of the color format) includes the gamuts of Red (R), Green (G), and Blue (B) (collectively RGB) and Cyan (C), Magenta (M), Yellow (Y), and Black (K) (collectively CMYK) color formats.

In the L* a* b* color format, color is viewed as point in three dimensional space, as defined by the three-dimensional coordinate system (L*, a*, b*), where each of the L* data, the a* data, and the b* data may correspond to individual color channels, and may therefore be referenced as channel data. In this three-dimensional coordinate system, the L* axis describes the brightness (luminance) of the color with values from 0 (black) to 100 (white). The a* axis describes the green or red ratio of a color with positive a* values (+a*) indicating red hue and negative a* values (−a*) indicating green hue. The b* axis describes the blue or yellow ratio of a color with positive b* values (+b*) indicating yellow hue and negative b* values (−b*) indicating blue hue. Generally, the values corresponding to the a* and b* axes may be unbounded, such that the a* and b* axes may include any suitable numerical values to express the axis boundaries. However, the a* and b* axes may typically include lower and upper boundaries that range from approximately −150 to 150. Thus, in this manner, each pixel color value may be represented as a three-tuple of the L*, a*, and b* values to create a final color for a given pixel.

As another example, a popular color format includes the red-green-blue (RGB) format having red, green, and blue channels. That is, in the RGB format, data of a pixel is represented by three numerical RGB components (Red, Green, Blue), that may be referred to as a channel data, to manipulate the color of pixel's area within the image. In some implementations, the three RGB components may be represented as three 8-bit numbers for each pixel. Three 8-bit bytes (one byte for each of RGB) may be used to generate 24-bit color. Each 8-bit RGB component can have 256 possible values, ranging from 0 to 255 (i.e., in the base 2 binary system, an 8-bit byte can contain one of 256 numeric values ranging from 0 to 255). This channel data (R, G, and B) can be assigned a value from 0 to 255 that can be used to set the pixel's color. For example, three values like (250, 165, 0), meaning (Red=250, Green=165, Blue=0), can denote one Orange pixel. As a further example, (Red=255, Green=255, Blue=0) means Red and Green, each fully saturated (255 is as bright as 8 bits can be), with no Blue (zero), with the resulting color being Yellow. As a still further example, the color black has an RGB value of (Red=0, Green=0, Blue=0) and white has an RGB value of (Red=255, Green=255, Blue=255). Gray has the property of having equal or similar RGB values, for example, (Red=220, Green=220, Blue=220) is a light gray (near white), and (Red=40, Green=40, Blue=40) is a dark gray (near black).

In this way, the composite of three RGB values creates a final color for a given pixel. With a 24-bit RGB color image, using 3 bytes to define a color, there can be 256 shades of red, and 256 shades of green, and 256 shades of blue. This provides 256×256×256, i.e., 16.7 million possible combinations or colors for 24 bit RGB color images. As such, a pixel's RGB data value indicates a degree of color or light each of a Red, a Green, and a Blue pixel is comprised of. The three colors and their intensity levels are combined at that image pixel, i.e., at that pixel location on a display screen, to illuminate a display screen at that location with that color. It is to be understood, however, that other bit sizes having fewer or more bits, e.g., 10-bits, may be used to result in fewer or more overall colors and ranges.

As a whole, the various pixels, positioned together in a grid pattern (e.g., pixel data 202a1p), form a digital image or portion thereof. A single digital image can comprise thousands or millions of pixels. Images can be captured, generated, stored, and/or transmitted in a number of formats, such as JPEG, TIFF, PNG and GIF. These formats use pixels to store or represent the image.

With reference to FIG. 2A, example image 202a1 illustrates a product (e.g., a tube of toothpaste) that appears in a typical location, e.g., bathroom or countertop space with the countertop and backsplash as a background or otherwise background feature. More specifically, image 202a1 comprises pixel data, including pixel data 202a1p defining a product region of product packaging (e.g., a tube or carton) of a product (e.g., toothpaste). Pixel data 202a1p includes a plurality of pixels including pixel 202a1p1, pixel 202a1p2, and pixel 202a1p3. In example image 202a1, each of pixel 202a1p1, pixel 202a1p2, and pixel 202a1p3 are representative of features of a product defining or otherwise corresponding to product data. Generally, in various embodiments, product data or otherwise product feature data may comprise one or more features identifiable with the pixel data of a given image. Each of these features may be determined from or otherwise based on one or more pixels in a digital image (e.g., image 202a1). For example, with respect to image 202a1, pixel 202a1p1 may be a relatively white pixel (e.g., pixels with relatively high RGB values across all RGB channels) positioned within pixel data 202a1p of the product packaging of the product (e.g., toothpaste) of FIG. 2A, which may be indicative of a brand name of toothpaste product as depicted in white text. The pixels may form a pattern in the shape of letters or styling indicative of the brand name.

In an example, pixel 202a1p2 may comprise relatively red pixels (e.g., pixels with high R (red) values in RGB based channels indicating a red color) and may be indicative of a typical color associated with the brand name of the product (e.g., the brand name toothpaste product is typically associated with the color red). As a further example, pixel 202a1p2 may also be part of a pattern of pixels defining an edge of the product packaging (e.g., tube of toothpaste or outer packaging, such as a carton containing a tube of toothpaste), which can be used to determine the shape of the product packaging, and therefore detect the product or otherwise product identifier. In some aspects, such shape, pattern, or edge may be used by a segmentation model to determine or detect what product or product identifier depicted in a given image.

As a further example, pixel 202a1p3 may comprise darker pixels (e.g., with lower values in the RGB based channels), which may be indicative of smaller font text that indicates a product variant, which defines a type of product (e.g., teeth whitening toothpaste). Such pixel features may comprise product data that may be used to train product-based learning model (e.g., train product-based learning model 108) to detect, based on the product data, a product or product identifier of a product.

In addition to pixels 202a1p1, 202a1p2, and 202a1p3, pixel data 202a1p includes various other pixels including remaining portions of the product packaging, including various other product data or information that may be analyzed and/or used for training of model(s), and/or analysis by use of already trained models, such as product-based learning model 108 and/or product-based learning model 108a as described herein. For example, pixel data 202a1p further includes pixels representative of features of SKUs, barcodes, QR codes, product text and/or other features identified in the pixel data and/or at a particular location in the image, where such pixels comprise unique identifiable features, which provides training information for detecting, based on such product data as identifiable in the pixel data, a product identifier of a product as described herein.

In addition, digital images of a products, as described herein, may depict various product features, which may be used to train a product-based learning model across a variety of different products having a variety of different product features. For example, as illustrated for images 202a1 and 202b1, the product features of these different can be different, where, for example the products have different shapes, labels, SKUs, barcodes, QR codes, brand names, product names, and the like.

FIG. 2B illustrates an example image 202a2 and its related pixel data that may be used for training and/or implementing a dosing learning model, in accordance with various embodiments disclosed herein. Example image 202a2 illustrates a product (e.g., a toothpaste) of a specific dosage (e.g., a toothpaste nurdle) and as depicted on a product implement (e.g., a toothbrush). More specifically, image 202a2 comprises pixel data, including pixel data 202a2p defining a product dosage region of a product (e.g., toothpaste). Pixel data 202a2p includes a plurality of pixels including pixel 202a2p1, pixel 202a2p2, and pixel 202a2p3. In example image 202a2, each of pixel 202a2p1, pixel 202a2p2, and pixel 202a2p3 is representative of features corresponding to the product or product implement, which may include attributes or features of the product and product implement, including, by way of non-limiting example, texture, composition, size, amount, etc. or the product and/or positioning of the product with respect to a product implement. Each of these features may be determined from or otherwise based on one or more pixels in a digital image (e.g., image 202a2). For example, with respect to image 202a2, pixel 202a2p1 comprises relatively pink pixels (e.g., pixels with medium R (red) values in RGB based channels), and may be indicate of a typical color associated with the product (e.g., the color of the toothpaste associated with the product or product brand may be typically pink in color; such color may come from the formulation or chemical composition of the product, e.g., toothpaste).

Pixel 202a2p2 comprises relatively white pixels (e.g., pixels with high RGB values in the RGB based channels) and may be indicate of a typical color associated with a product implement (e.g., a toothbrush). As a further example, pixel 202a2p2 may further be part of a pattern of pixels indicating a shape or otherwise edge of the product implement (e.g., an elongated pattern of pixels representing a toothbrush). Such pattern of pixels may also indicate a shape or edge of a product appliance (e.g., a set of dentures) (not shown). Such shape, pattern, or edge may be used by a segmentation model to determine or detect a product, product dosage, product implement, and/or product appliance within the image.

Pixel 202a2p3 may comprise darker pink pixels (e.g., with lower R values in the RGB based channels), indicating a portion of the product dosage (e.g., toothpaste nurdle) that has sagged or flowed, which may be indicative of a characteristic of the product dosage, such as viscosity of the toothpaste, which may indicate its relative efficacy, size, shape, or amount. Such pixel features may comprise product data that may be used to train a dosing learning model (e.g., train dosing learning model 109 and/or dosing learning model 109a) to output, based on a dosage comparison to a target dosage, a feedback indication designed to address at least one feature identifiable within the pixel data comprising the dosage of the product. The target dosage itself may be based on representative training images depicting ideal or ground truth base sizes, amounts, etc. of a given product dosage of a given product, and/or features of the user, such as a user's dental arch. In an example, the target dosage of the product is defined at least in part by a dental arch state. The dental arch state may be determined based on an analysis of at least one dental arch image. For example, if a dental arch image shows stains or plaque buildup, the target dosage may include different dosing at different times based on implement and/or product.

In addition to pixels 202a2p1, 202a2p2, and 202a2p3, pixel data 202a2p includes various other pixels including remaining portions of the product dosage and product implement, including various other pixel based information that may be analyzed and/or used for training of model(s), such as dosing learning model 108 as described herein. For example, pixel data 202a2p further includes pixels representative of features of physical attributes of the product, product implement, and/or product appliance, and/or other features identified in the pixel data and/or at a particular location in the image, where such pixels comprise unique identifiable features, which provides training data for the dosing learning model as described herein.

A digital image, such as a training image, an image as submitted by users, or otherwise a digital image (e.g., any of images 202a1, 202a2, 202b1, and/or 202b2), may be or may comprise a cropped image. Generally, a cropped image is an image with one or more pixels removed, deleted, or hidden from an originally captured image. In some aspects, each image of the one or more of the plurality of training images e.g., any of images 202a1, 202a2, 202b1, and/or 202b2) or the image of a product and/or product dosage comprises at least one cropped image depicting the product or product dosage having a given feature. For example, with reference to FIG. 2A, cropped portion 202a1c1 represents a first cropped portion of image 202a1 that removes portions of the background 202a1b or non-product features (outside of cropped portion 202a1c1) that may not include readily identifiable regions that have a product and/or product features. As a further example, cropped portion 202a1c2 represents a second cropped portion of image 202a1 that removes further portions of the image (outside of cropped portion 202a1c2) that includes additional background or non-product features compared to the cropped portion 202a1c1, and therefore reduces the amount of pixels and data that the give system (e.g., system 100) must store or otherwise analyze as training data.

Similarly, with reference to FIG. 2B, cropped portion 202a2c1 represents a first cropped portion of image 202a2 that removes portions of the background or non-product features (outside of cropped portion 202a2c1) that may not include readily identifiable regions that have a product, product dosage, product implement, and/or otherwise product features. As a further example, cropped portion 202a2c2 represents a second cropped portion of image 202a2 that removes further portions of the image (outside of cropped portion 202a2c2) that includes additional background or non-product features compared to the cropped portion 202a2c1, and therefore reduces the amount of pixels and data that the give system (e.g., system 100) must store or otherwise analyze as training data. In various embodiments, analyzing and/or use of cropped images for training yields improved accuracy of a training the learning models (e.g., product-based learning model and/or dosing learning model). It also improves the efficiency and performance of the underlying computer system in that such system processes, stores, and/or transfers smaller size digital images. Still further, images may be sent as cropped or that otherwise include extracted or depicted product and/or product dosage without depicting personal identifiable information (PII) of a user. In some aspects, each image of a plurality of training images may comprise at least one cropped image removing at least a portion of PI of a user. For example, a cropping algorithm automatically crops each item in the image (if more than one is presented), to check and crop out human/facial accidental images (e.g., a mirror reflection) and remove such PII data. Such cropped images provide a security improvement, i.e., where the removal of PI provides an improvement over prior systems because cropped or redacted images, especially ones that may be transmitted over a network (e.g., the Internet), are more secure without including PII information of a user. Importantly, the systems and methods described herein may operate without the need for such non-essential information and thus is able to operate with smaller data size images, which provides an improvement, e.g., a security and a performance improvement, over conventional systems. Moreover, while FIGS. 2A and 2B may depict and describe a cropped image, it is to be understood, however, that other image types including, but not limited to, original, non-cropped images (e.g., original image 202a1 and/or original image 202a2) and/or other types/sizes of cropped images may be used or substituted as well.

In various embodiments, digital images (e.g., images 202a1, 202a2, 202b1, 202b2), whether used as training images depicting products, implements, and/or appliances, or used as images depicting product dosages by specific users, may comprise multiple angles or perspectives depicting the products and/or product dosages of each of the respective individual or the user. That is, each image of the one or more of the plurality of training images or the image of a user may comprise multiple angles or perspectives depicting products, product dosages, product implements, product appliances, and/or other features as described herein. The multiple angles or perspectives may include different views, angular positions, zoomed positions of the user and/or backgrounds, lighting conditions, and/or otherwise environments in which a given product, product dosage, product implement, and/or product appliance is positioned within a given image. For example, FIG. 1 includes images (e.g., 202a1, 202a2, 202b1, 202b2) that depict products, product packaging, product dosages, and/or product implements that were captured using different lighting conditions (e.g., visible, UV) at different angles. Such images may be used for training AI models (e.g., product-based learning model and/or dosing learning model), or for analysis, and/or feedback indication(s) designed to address at least one feature identifiable within the pixel data comprising the dosage of the product, as described herein.

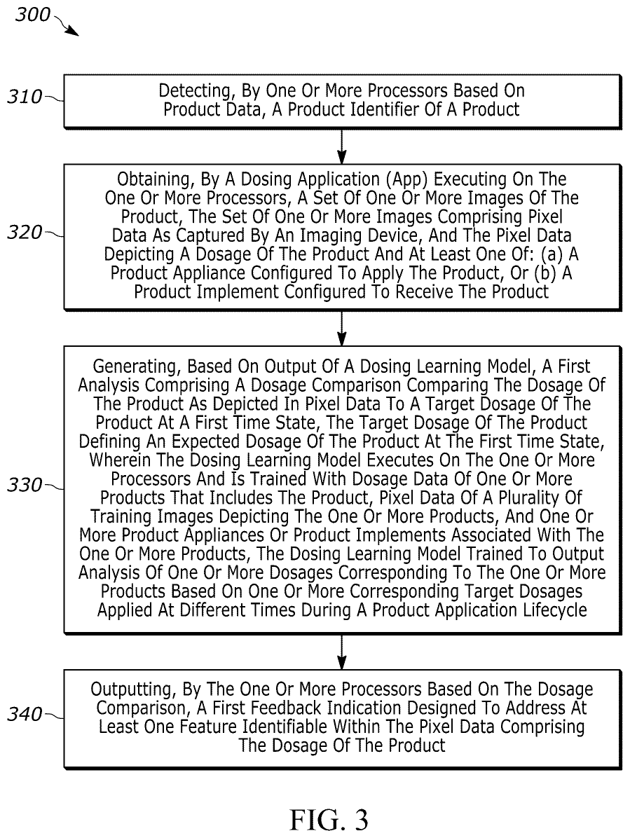

FIG. 3 illustrates an example digital imaging and AI-based method 300 for analyzing product dosing, in accordance with various embodiments disclosed herein. Method 300 may be implemented by one or more processors, including processor 104, a processor of a mobile device (e.g., such as computing device 1l1c1), or multiple processors, such as processor 104 and a processor of a mobile device (e.g., such as computing device 111c1) communicating across computer network 120.

At block 310, method 300 comprises detecting, by one or more processors based on product data, a product identifier of a product. In various aspects, a product may comprise an oral product composition, including, for example a toothpaste, an emulsion, a gel, a mouth rinse, a mouthwash, and/or a dental adhesive. However, it should be understood, that the disclosure herein could relate to other products, including non-oral products or otherwise different products, such as skin care products, shaving products, and/or other consumer products.

In some aspects, the detection of a given product identifier may be based on product data comprising information provided to the dosing app (e.g., dosing app 107) by a user. For example, in some aspects, a user may input, into a graphic user interface (GUI) (e.g., as described herein for FIG. 5) an identifier, description, or otherwise indication of what product is being used. The user may also input other information, including, by way of non-limiting example, a product implement and/or product appliance is being used.

Still further, in various aspects, a product identifier can be submitted (e.g., to dosing app 107 and/or server 102) as an input to look up or link to additional data defining the product. Such additional data may include a formula specification of the product (e.g., the chemical formula of a toothpaste or mouthwash), traits of the products (e.g., the texture or viscosity of a cream, toothpaste, gel, etc.), the packaging data of the product (e.g., the shape, size, such as ounces, color, markings, descriptions, or other characteristics of a given product), and/or clinical indications of the product (e.g., the active ingredient of a product, the expected effect of the product at certain amounts or dosages, and/or the expected efficacy of a product at certain amounts or dosages). More generally, with a unique product identified (e.g., by way of detecting the given product identifier), dosing app 107 and/or server 102 can look-up and link to other data sources where different information is provided about the product. This includes, but is not limited to, formulation specifications, product traits, packaging traits, clinical indications, etc. This additional data can comprise data package, or otherwise meta data, about a given product that augments data collection and identification efforts of the product and allows identification of the product with little or no input by the user. In addition, such additional data (e.g., data package and/or meta data) can be provided, e.g., via input by a user or programmatically upon detection within pixel data, to assist with identifying related products, e.g., a toothpaste related product, once identified, can be used to detect a product implement such as a toothbrush and/or a product appliance, such as dentures. Such cross identification can be implemented, for example, where such products and product implements/product appliances are correlated product (e.g., typically used together). In addition, such additional data (e.g., data package and/or meta data) may also be provided to the dosing learning model (e.g., dosing learning model 109 and/or dosing learning model 109a) as described herein to improve its output and/or prediction related to the feedback it provides regarding dosing, for example, as described herein.

Additionally, or alternatively, in some aspects a product identifier may be as detected by a product-based learning model. For example, with reference for FIG. 1, method 300 may implement product-based learning model 108 and/or product-based learning model 108a. The product-based learning model (e.g., product-based learning model 108 and/or product-based learning model 108a) is an image (or video) based model or algorithm that allows a user to capture a digital image of one or more products, product packages, product implements, product appliances, and/or other features as described herein, each of which can be in an isolated space or in a broad context space (e.g., a bathroom counter surrounded by other non-related products/implements/devices). Further, the image(s) may comprise pixel data of at least a portion of a product dosage, a product implement and/or a product application, for example, as described herein.

In various aspects, the one or more processors may comprise processor 104 of server 102. Additionally, or alternatively, the one or more processors may comprise a processor of a mobile device (e.g., computing device 111c1). Images, as used with method 300, and more generally as described herein, are pixel-based images as captured by an imaging device (e.g., an imaging device of user computing device 111c1). In some embodiments an image may comprise or refer to a plurality of images such as a set of images (e.g., frames) as collected using a digital video camera. Frames comprise consecutive images defining motion, and can comprise a movie, a video, or the like.

In various aspects, the product-based learning model (e.g., product-based learning model 108 and/or product-based learning model 108a) can comprise an artificial intelligence (AI) based model trained with at least one AI algorithm. Training of the product-based learning model involves image analysis of the training images to configure weights of the product-based learning model, and its underlying algorithm (e.g., machine learning or artificial intelligence algorithm) used to predict and/or classify additional images. For example, in various embodiments herein, generation of the product-based learning model involves training the product-based learning model with the plurality of training images (e.g., images 202a1, 202a2, 202b1, 202b2), each of which may depict a plurality of products, product implements, product appliances, background features, and/or other features described herein, where each of the training images comprise pixel data and depict features of the product and/or these product related features.

In some aspects, one or more processors of a server or a cloud-based computing platform (e.g., imaging server 102) may receive the plurality of training images (e.g., images 202a1, 202a2, 202b1, 202b2) of the plurality of products via a computer network (e.g., computer network 120). In such embodiments, the server and/or the cloud-based computing platform may train the product-based learning model (e.g., product-based learning model 108 and/or product-based learning model 108a) with the pixel data of the plurality of training images. Additionally, in some aspects, the product-based learning model may be further trained with additional data (e.g., packaged data or meta data), such as text data or product information data including, for example, product brand names, slogans, product labeling, SKUs, or otherwise as described herein as related to a product, product implement, ad/or applications. In such aspects, predictions or otherwise output, as generated by the product-based learning model, may be based on such text data or product data.

In various aspects, a machine learning imaging model, such as the product-based learning model and/or the dosing learning model, may be trained using a supervised or unsupervised machine learning program or algorithm. The machine learning program or algorithm may employ a neural network, which may be a convolutional neural network, a vision transformer, a deep learning neural network, or a combined learning algorithm or program that learns based on features or feature datasets (e.g., pixel data) in a particular areas of the image of interest. The machine learning programs or algorithms may also include natural language processing, semantic analysis, automatic reasoning, regression analysis, support vector machine (SVM) analysis, decision tree analysis, random forest analysis, K-Nearest neighbor analysis, naïve Bayes analysis, clustering, reinforcement learning, and/or other machine learning algorithms and/or techniques. In some embodiments, the artificial intelligence and/or machine learning based algorithms may be included as a library or package executed on imaging server 102. For example, libraries may include the TENSORFLOW based library, the PYTORCH library, and/or the SCIKIT-LEARN Python library.

Machine learning may involve identifying and recognizing patterns in existing data (such as identifying features of a given product, product implement, and/or product appliance, in the pixel data of image as described herein) in order to facilitate making predictions, classifications, or identification for subsequent data (such as using the model on new pixel data of a new image in order to detect a product identifier within the pixel data and/or determine or generate a feedback indication designed to address at least one feature identifiable within the pixel data comprising the dosage of the product).

Machine learning model(s), such as the product-based learning model and/or the dosing learning model, as described herein for some embodiments, may be created and trained based upon example data (e.g., training data and related pixel data) inputs or data (which may be termed “features” and “labels”) in order to make valid and reliable predictions for new inputs, such as testing level or production level data or inputs. In supervised machine learning, a machine learning program operating on a server, computing device, or otherwise processor(s), may be provided with example inputs (e.g., “features”) and their associated, or observed, outputs (e.g., “labels”) in order for the machine learning program or algorithm to determine or discover rules, relationships, patterns, or otherwise machine learning “models” that map such inputs (e.g., “features”) to the outputs (e.g., labels), for example, by determining and/or assigning weights or other metrics to the model across its various feature categories. Such rules, relationships, or otherwise models may then be provided subsequent inputs in order for the model, executing on the server, computing device, or otherwise processor(s), to predict, based on the discovered rules, relationships, or model, an expected output.

In unsupervised machine learning, the server, computing device, or otherwise processor(s), may be required to find its own structure in unlabeled example inputs, where, for example multiple training iterations are executed by the server, computing device, or otherwise processor(s) to train multiple generations of models until a satisfactory model, e.g., a model that provides sufficient prediction accuracy when given test level or production level data or inputs, is generated.

Supervised learning and/or unsupervised machine learning may also comprise retraining, relearning, or otherwise updating models with new, or different, information, which may include information received, ingested, generated, or otherwise used over time. The disclosures herein may use one or both of such supervised or unsupervised machine learning techniques.

In various aspects, the product-based learning model and/or the dosing learning model may be trained, by one or more processors (e.g., one or more processor (e.g., processor 104) of server 102 and/or processors of a computer user device, such as a mobile device) with the pixel data of a plurality of training images (e.g., any of images 202a1, 202a2, 202b1, and/or 202b2). In various aspects, the product-based learning model is configured or otherwise trained to output product predictions of one or more product identifiers corresponding to the one or more products depicted within the pixel data of the plurality of training images. For example, in some aspects, the one or more product identifiers are based on prediction(s) of the product-based learning and detection model, where the model predictions predict, with a degree of predictive accuracy, how likely it is that a given product (and/or product implement and/or application) depicted in one or more images is actually the product (and/or product implement and/or application) actually depicted. The prediction(s) may be based on pixel data and features identified in images as described, for example, by FIGS. 2A and 2B herein.

In various aspects, the product-based learning model and/or the dosing learning model is accessible by dosing app 107 and/or dosing app 107a, and can be implemented on a computing device (e.g., computing device 111c1) and/or server 102, where computing instructions, implementing on, one or more processors of the computing device (e.g., computing device 111c1) and/or server 102 obtain a set of one or more images of a product. In such aspects, the product data comprises the set of one or more images of the product. Alternatively or additionally, the product data may comprise data inputted by the user. Further, the set of one or more images (e.g., any of images 202a1, 202a2, 202b1, and/or 202b2) of the product comprises pixel data of the product as captured by an imaging device. The pixel data of the product may depict least a portion of the product, product implement, product appliance, or other features described herein.

The product-based learning model (e.g., the product-based learning model 108 and/or the product-based learning model 108a) may then detect, based on output (e.g., a product prediction output by the model) of the product-based learning model inputting the pixel data of the product, the product identifier of the product. In various aspects, one or more product identifiers as output by the product-based learning model may be based one or more features identifiable within the pixel data of the plurality of training images. Such features may comprise, by way of none limiting example, pixel data defining or forming pixel patterns within image(s) defining a product category (e.g., oral care or skin care) of the one or more products; a product brand of the one or more products; a product variant (e.g., a specific type or formulation) of the one or more products; a product form (e.g., a texture, viscosity, pattern) of the one or more products, and/or a product packaging (e.g., a shape, color, or size (e.g., ounces)) of the one or more products. For example, the product-based learning model may detect a franchise or category of a product, which may comprise a segment of a product market that the product belongs to (e.g., oral care such as toothbrushes, oral care—implements, and/or skin care—facial cleansers). As a further example, the product-based learning model may detect a brand of a product this is determined from pixel data of text or logo image processing from the image. As a further example, the product-based learning model may detect a product variant, which may be determined from analyzed text detected in the image. As a further example, the product-based learning model may detect a form of the product, which may comprise, for example, one or more physical properties of the product, product implement, and/or product appliance (e.g., a liquid, a gel, a powder, a toothbrush, etc.). As a further example, the product-based learning model may detect the product packaging of a product, which may comprise, e.g., a tube, a box, a bag, etc. The product-based learning model may be trained to detect one or all of these the features in the pixel data to narrow and predict a unique product, product implement, and/or product appliance.

In some aspects, the product-based learning model (e.g., the product-based learning model 108 and/or the product-based learning model 108a) may be trained to output a product prediction defining a percentage accuracy that the product identifier correctly identifies the product. In some aspects, the percentage accuracy may comprise 90% or greater. For example, the product-based learning model has been tested to have a greater than 90% prediction and detection accuracy across a several consumer product categories, including oral care, including for over 300 oral care products, product implements, and/or product appliances.

Still further, in some aspects, the product-based learning model (e.g., the product-based learning model 108 and/or the product-based learning model 108a) can be further trained to filter or distinguish one or more background features (e.g., a bathroom countertop or backsplash) or background products (e.g., non-products or products other than those being imaged or otherwise targeted) from the one or more products and corresponding one or more product identifiers depicted in the pixel data of the plurality of training images. In such aspects, the product-based learning model is trained to detect a given product or portion thereof against background related features (e.g., a cabinet background or one or more non-products or non-interest products) by training the product-based learning model to identify and detect such background features or background products for the removal or distinguishing of such background features or background products in the pixel data.

With further reference to FIG. 3, at block 320, method 300 comprises obtaining, by a dosing app (e.g., dosing app 107 and/or dosing app 107a) executing the one or more processors, a set of one or more images (e.g., images 202a2 and/or 202b2) of the product. The set of one or more images may comprise pixel data as captured by an imaging device (e.g., a mobile device such as computing device 111c1). More generally, the set of one or more images may comprise digital image(s) (e.g., 202a2 and/or 202b2) as captured by an imaging device (e.g., a digital camera of the mobile device 111c1).

In various aspects, the pixel data as captured by an imaging device (e.g., a mobile device such as computing device 111c1) may depict a dosage of the product. Further, the pixel data may also include at least one of: (a) a product appliance configured to apply the product, or (b) a product implement configured to receive the product. In some example aspects, a product implement may comprise at least one of a manual toothbrush, a battery powered toothbrush, an electrical rechargeable toothbrush, a brush head, a toothbrush refill, a rinsing cup, a tongue scraper, a tongue cleaner, an applicator wand, a tray, and/or other oral care products or product components. It is to be understood, however, that additional and/or different product implements, including of additional and/or different product categories (e.g., skin care) are also contemplated herein.

In additional and/or alternative example aspects, a product appliance may comprise at least one of a partial denture, a full denture, a bridge, and/or a retainer. It is to be understood, however, that additional and/or different product appliances, including of additional and/or different product categories (e.g., skin care) are also contemplated herein.

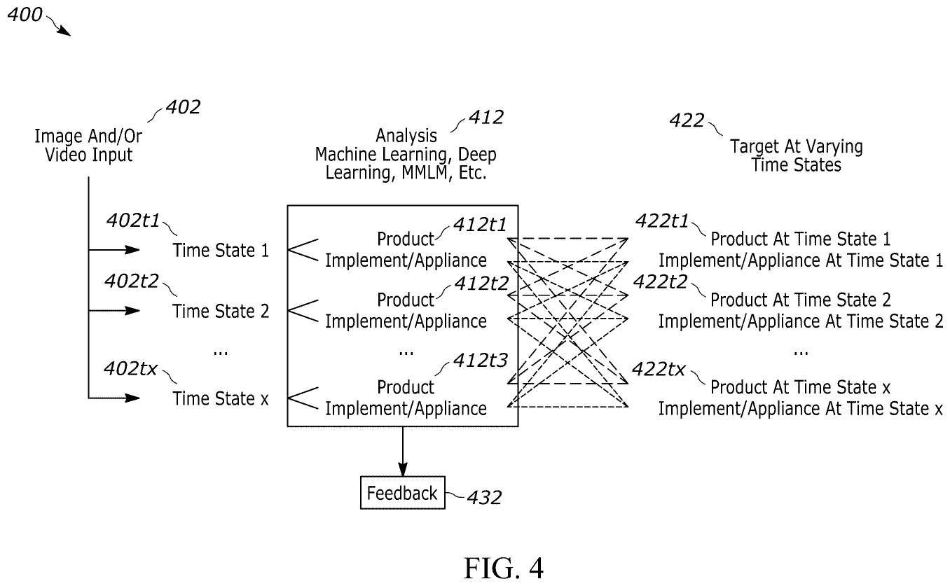

With further reference to FIG. 3, at block 330, method 300 comprises generating, based on output of a dosing learning model (e.g., dosing learning model 109), a first analysis comprising a dosage comparison comparing the dosage of the product (e.g., a nurdle of toothpaste) as depicted in pixel data to a target dosage of the product at a first time state. The first state may comprise one of several states as shown and described for FIG. 4 or otherwise as described herein. The dosage comparison may comprise a comparison of the size, amount, texture, or other attribute or characteristic as described herein of the product to a target dosage of the product. In various aspects, the target dosage of the product (e.g., toothpaste) may define an expected dosage (e.g., expected toothpaste nurdle or otherwise dimension) of the product at the first time state. In various aspects, the dosing learning model is trained to output an analysis of one or more dosages corresponding to the one or more products based on one or more corresponding target dosages applied at different times (e.g., different time states as shown and described for FIG. 4) during a product application lifecycle.

In the example of method 300, the dosing learning model (e.g., dosing learning model 109 and/or dosing learning model 109a) executes on one or more processors and is trained with various training data. Such training data may comprise dosage data of one or more products that includes the product. For example, the dosage data of one or more products may comprise at least one of: (a) an amount, size, or dimension of the product; (b) an amount, size, or dimension of the product relative to the product appliance and/or the product implement; and/or (c) a composition of the product (e.g., such as an ingredient level and/or density of the product).

Further, the training data used to train dosing learning model (e.g., dosing learning model 109 and/or dosing learning model 109a) may comprise pixel data of a plurality of training images depicting the one or more products, and one or more product appliances or product implements associated with the one or more products. In some aspects, the plurality of training images used for training the dosage learning model comprises training images depicting one or more dosages of the one or more products such that the dosage learning model is trained to correlate the one or more dosages depicted in pixel data of the plurality of training image (e.g., such as shown and describe for FIG. 2B herein) to the dosage data of one or more products. The plurality of training images may include images of the specific product the user is analyzing with the dosing learning model or it may not include the images of the specific product the user is analyzing with the dosing learning model. The dosing learning model may be able to compare the dosage of the specific product to a target dosage of the product, for example, based on other product data and the training images of other products.

In some aspects, the dosing learning model (e.g., dosing learning model 109 and/or dosing learning model 109a) comprises a segmentation model trained to identify segmented portions within the image (e.g., segments, such as segments of the product, product dosage, or other features defined herein) and to generate a segmentation mapping defining, in the pixel data, the dosages of the product and the product appliance or the product implement configured to apply the dosages. In such aspects, the dosing comparison may be generated by comparing one or more segmented portion(s) of pixels defining the dosage of the product defined in the segmentation mapping to a predefined set of pixels defining the target dosage of the product. For example, a segmentation model implemented by dosing learning model 109 and/or dosing learning model 109a may analyze pixel edges, pixel shapes, or otherwise pixel patterns to detect and/or segment a product dosage from a given image. The dosage size, amount, texture, etc. can be determined from a segmented set of pixels defining the product dosage. The segmented set of pixels can then be compared or analyzed against a given set of pixels or otherwise information defining target dose of the product dosage, and, in some cases, at a given time of use or lifecycle of the product dosage.

With further reference to FIG. 3, at block 340, method 300 further comprises outputting, by the one or more processors based on the dosage comparison, a feedback indication designed to address at least one feature identifiable within the pixel data comprising the dosage of the product. The feedback indication may comprise, e.g., a visual or audio feedback indication, e.g., as output by a computing device (e.g., computing device 111c1/) depicting, defining, or otherwise indicating the actual versus target dosage of the product. In various aspects, a feedback indication can further comprise at least one of: (a) a qualitative rating (e.g., an ordinary rating such as “poor,” “fair,” “good,” “excellent” with respect to the target dosage); (b) a numeric assessment (e.g., a percentage correlating the percentage of the actual dosage to the target dosage); (c) a visual projection (e.g., a display on a GUI of a graphic indicating correlation to the target dosage); (d) an augmented reality annotation (e.g., a superimposed image of the actual dosage to the target dosage); (e) and/or a categorical rating to the target dosage (e.g., a scale value, e.g., 0-10 where 10 defines a perfect correlation and where 0 defines no correlation).

In some aspects, a feedback indication may be generated based on the dosing comparison and additional data. For example, in some aspects the additional data may comprise data of the target dosage. The target dosage itself, and comparison thereto of an actual dosage depicted in an image, can depend on the amount, size, dimension of given the product applied, e.g., either absolute size or size relative to an implement, and, in some aspects, may also depend on the composition of the product (e.g., ingredient level, density, or other such physical and/or chemical properties of the product).

Additionally, or alternatively, the additional data that the feedback indication may be based on may include at least one of: (a) a physical attribute of the product appliance or the product implement; (b) a pattern or arrangement of the product as positioned on or with respect to the product appliance or the product implement; and/or (c) a provision of the dosage of the product by a user when applying the product with the product appliance or the product implement (e.g., a pattern of toothpaste on a toothbrush used when a user is brushing his or her teeth). Such additional data may be detected with pixel data of images provided to the dosing learning model and/or as provided as input by a user to dosing app 107 and/or dosing app 107a.

FIG. 4 illustrates an example digital imaging and AI-based method 400 for analyzing product dosing including analyzing product dosing across a plurality of time states, in accordance with various embodiments disclosed herein. Method 400 may be implemented in the same manner as described herein for method 300, where method 400 further illustrates implementation of analyzing product dosing across a plurality of time states (e.g., first time state 402t1, second time state 402t2, and further time state(s) 402tx). Generally, any of the given time states can represent a new time state, before use time state, during use time state, and/or last/latest time state, each defining a state of time at which a give product and/or product dosage is used, applied, or otherwise imaged by a user. For example, a user can provide new, updated, or further images of the product and/or product dosages at various times during applying or using a product, where adjustments to such product usage can be made by the user over time. For example, the user can reassess product usage over various states of time until accuracy is achieved. This reassessment can occur within one treatment (e.g., the user can add more toothpaste to the brush after the first analysis) or over several treatments.

As shown for FIG. 4, computing instructions of the dosing app, when executed by one or more processors (e.g., one or more processors of a computing device and/or server 102), may cause the one or more processors to obtain input 402 of a set of one or more images of the product. For example, input 402 of a set of one or more images may comprise a first set of images at first time state 402t1, a second set of images at second time state 402t2, and/or a further set of images at time state 402tx. It is to be understood that the further set of images may represent a third set of images at a third time state, a fourth set of images at a fourth time state, and so on.

The set of images as input 402 at the various time states may comprise pixel data (e.g., second pixel data at second time state 402t2) as captured by the imaging device (e.g., a camera of computing device 111c1). Such pixel data (e.g., second pixel data) may depict a dosage (e.g., a second dosage) of the product and the product appliance or the product implement at the given time state (e.g., second time state 402t2).

In the input 402 of the set of images at the various time states may be provided to a machine learning model 412, such as dosing learning model 109 and/or 109a, which may implement deep learning, e.g., a model trained with various weights on the features identifiable with the set of images. The dosing model may generate output for a given time state. For example, at the second time state 402t2, the dosing model may analyze (at 412t2) the images of a product and/or product implement and/or appliance generate output of a second analysis comprising a second dosage comparison of the second dosage of the product as depicted in second pixel data to a second target dosage of the product at a second time state. At 422t2, the second target dosage of the product may define a second expected dosage of the product at the second time state 402t2. It is to be understood that other output (e.g., 422t1 and/or 422tx) may define other expected dosages, products, product implements, product appliances, etc. as other time states (e.g., at 402t1 and/or 402tx, respectively).

Additional feedback indications 432 may then be output for each of the various time states. For example, for the second time state 422t2, a second feedback indication may be output based on the second dosage comparison designed to address at least one feature identifiable within the second pixel data comprising the second dosage of the product at the second time state.

FIG. 5 illustrates an example user interface 502 as rendered on a display screen 500 of a user computing device in accordance with various embodiments disclosed herein. For example, as shown in the example of FIG. 5, user interface 502 may be implemented or rendered via an application (app) executing on user computing device 111c1. User interface 502 may be implemented or rendered via a native app executing on user computing device 111c1. In the example of FIG. 5, user computing device 111c1 is a user computer device as described for FIG. 1, e.g., where 111c1 is illustrated as an APPLE IPHONE that implements the APPLE iOS operating system and that has display screen 500. User computing device 111c1 may execute one or more native applications (apps) on its operating system, including, for example, dosing app as described herein. Such native apps may be implemented or coded (e.g., as computing instructions) in a computing language (e.g., SWIFT) executable by the user computing device operating system (e.g., APPLE iOS) by the processor of user computing device 111c1.

Additionally, or alternatively, user interface 502 may be implemented or rendered via a web interface, such as via a web browser application, e.g., SAFARI and/or GOOGLE CHROME app(s), or other such web browser or the like.

As shown in the example of FIG. 5, user interface 502 comprises a graphical representation (e.g., of image 202a1 or portion thereof) of a product (e.g., toothpaste). Image 202a1 may comprise the image of the product (or graphical representation thereof) comprising pixel data (e.g., pixel data 202a1p) of at least a portion of the product as described herein. In the example of FIG. 5, graphical representation or image (e.g., image 202a1) of the product is annotated with one or more graphics (e.g., augmented reality annotation 202a1AR) and textual rendering(s) (e.g., text 202a1t) corresponding to various features identifiable within the pixel data comprising a product or product dosage. For example, the area of pixel data of the product or product dosage may be annotated or overlaid on top of the image (e.g., image 202a1) to highlight the area or feature(s) identified within the pixel data (e.g., feature data and/or raw pixel data) by the dosing learning model (e.g., dosing learning model 109). In various embodiments, the pixels identified as the specific features (e.g., any one of pixels 202a1p1-202a1p3), may be highlighted or otherwise annotated when rendered on display screen 500.

In various aspects, a feedback indication may be rendered on a display screen (e.g., display screen 500) to indicate (e.g., graphically indicate in the example of FIG. 5) a difference or similarity between the dosage of the product and the target dosage of the product. Still further, additionally or alternatively, a target dosage may be rendered or otherwise comprise at least a visual appearance of the product, a color of the product, a volume of the product, an amount of the product, a dimension of the product, a pattern of the product, a shape of application of the product, a texture of the product, a density of the product, a relative ratio of the product, and/or a position of the product relative to the product appliance or the product implement.

As shown by way of example for FIG. 5, an augmented reality annotation 202a1AR is shown as a superimposed image of a target dosage on top of the actual dosage (e.g., a toothpaste nurdle). In this way, the target dosage augmented reality annotation 202a1AR graphically and visually illustrates and compares the target dosage to the actual dosage, e.g., at a given time state. Similarly, textual rendering (e.g., text 202a1t) shows a numeric assessment (e.g., a percentage value of 73% correlating the percentage of the actual dosage to the target dosage), which may indicate that the pixel(s) detected for the actual dosage are 73% the amount of pixels of the target dosage for the given time state. It is to be understood that other graphical and/or textual rendering types or values are contemplated herein, where graphical and/or textual rendering types or values may be rendered, for example, such as additional and/or different graphics or text to describe or illustrate the dosage comparison between the actual and target dosages.

User interface 502 may also include or render a feedback indication 510 in the form of a message 510m. In the embodiment of FIG. 5, the feedback indication 510 comprises a message 510m to the user designed to indicate that the actual dosage amount detected is of a low dosage amount at only 73% of the size of the target dosage amount.

User interface 502 may also include or render a dosage recommendation 512. For example, the dosing app may render, on a display screen of a computing device, at least one dosage recommendation based on the detected product dosage and/or otherwise feedback indication. In various aspects, the dosage recommendation may comprise a textual recommendation, an imaged based recommendation, and/or virtual rendering of the product and/or product dosage, and/or an augmented reality (AR) based recommendation rendered in a proximity to or superimposed on the display screen with the product (e.g., as shown for 202a1AR). Further, a dosage recommendation can be displayed on the display screen 500 of the computing device with instructions for adjusting the dosage identifiable in the pixel depicting the dosage of the product. For example, in the embodiment of FIG. 5, dosage recommendation 512 comprises a message 512m to the user designed to address at least one feature identifiable within the pixel data comprising the product and/or product dosage. As shown in the example of FIG. 5, message 512m recommends to the user to increase the dosage amount of the identified product.

In various aspects, a dosage recommendation may comprise a product recommendation for a manufactured product. For example, message 512m also includes a product recommendation that may have increased efficacy for the user, e.g., extra whitening for a toothpaste related product, which may have a greater efficacy for the user at lower dosages (e.g., the 73% dosage that the user may typically use). The product recommendation can be correlated to the identified feature within the pixel data (e.g., a low dosage), and the user computing device 111c1 and/or server 102 can be instructed to output the product recommendation when the feature (e.g., low dosage amount) is identified or classified.

In the example of FIG. 5, user interface 502 renders or provides a recommended product (e.g., manufactured product 524r) as determined by dosing model (e.g., dosing learning model and/or product-based learning model) and its related image analysis of image 202a1 and its pixel data and various features. In the example of FIG. 5, this is indicated and annotated (524p) on user interface 502.

User interface 502 may further include a selectable UI button 524s to allow the user (e.g., the user of image 202a1) to select for purchase or shipment the corresponding product (e.g., manufactured product 524r). In some embodiments, selection of selectable UI button 524s may cause the recommended product(s) to be shipped to the user and/or may notify a third party that the individual is interested in the product(s). For example, either user computing device 111c1 and/or imaging server 102 may initiate, based on the feedback indication 510 and/or the dosage recommendation 512, the manufactured product 524r (e.g., extra whitening toothpaste) for shipment to the user. In such aspects, the product can be packaged and shipped to the user.

In various embodiments, a graphical representation or image (e.g., image 202a1), with graphical annotations (e.g., area of pixel data 202a1p), textual annotations (e.g., text 202a1t), and the annotation 202a1AR and the dosage recommendation 512 may be transmitted, via the computer network (e.g., from an imaging server 102 and/or one or more processors) to user computing device 111c1, for rendering on display screen 500. In other embodiments, no transmission to the imaging server of the user's specific image occurs, where such information or data may instead be generated locally, by the dosing learning model 109a executing and/or implemented on the user's mobile device (e.g., user computing device 111c1) and rendered, by a processor of the mobile device, on display screen 500 of the mobile device (e.g., user computing device 111c1).

In some embodiments, a dosage recommendation may be rendered on the display screen 500 in real-time or near-real time, during, or after receiving, the set of images. For example, the dosage recommendation can be displayed on the display screen of the computing device with instructions for treating, with the manufactured product, the at least one feature identifiable in the pixel data comprising the dosage of the product. Still further, any one or more of graphical representations (e.g., image 202a1), with graphical or textual annotations (e.g., 202a1t and/or 202a1AR), or other information shown for FIG. 5, may be rendered (e.g., rendered locally on display screen 500) in real-time or near-real time during or after receiving, the image of the product and/or product dosage. In embodiments where the image is analyzed by imaging server 102, the image may be transmitted and analyzed in real-time or near real-time by imaging server 102.

In some embodiments, the user may provide a new image that may be transmitted to imaging server 102 for updating, retraining, or reanalyzing by dosing learning model and/or product-based learning model. In other embodiments, a new image that may be locally received on computing device 111c1 and analyzed, by dosing learning model and/or product-based learning model, on the computing device 111c1.

In addition, as shown in the example of FIG. 5, the user may select selectable button 512i for reanalyzing (e.g., either locally at computing device 111c1 or remotely at imaging server 102) a new image at new or otherwise additional time state (e.g., as shown for FIG. 4 herein). Selectable button 512i may cause user interface 502 to prompt the user to attach for analyzing a new image. Imaging server 102 and/or a user computing device such as user computing device 111c1 may receive a new image comprising pixel data of a product or product dosage. The new image can be captured by the imaging device. The new image (e.g., image 202a1) may comprise pixel data of a product, product dosage, product implement, or other feature(s) as described herein. The dosing learning model, executing on the memory of the computing device (e.g., imaging server 102), may analyze the new image captured by the imaging device to determine a product, product dosage, product identifier, or other aspects as described herein. The computing device (e.g., imaging server 102) may output, based on a dosage comparison of the image and the second image, a further feedback indication, for example, as described for FIG. 4 or elsewhere herein.

### ADDITIONAL CONSIDERATIONS

Although the disclosure herein sets forth a detailed description of numerous different embodiments, it should be understood that the legal scope of the description is defined by the words of the claims set forth at the end of this patent and equivalents. The detailed description is to be construed as exemplary only and does not describe every possible embodiment since describing every possible embodiment would be impractical. Numerous alternative embodiments may be implemented, using either current technology or technology developed after the filing date of this patent, which would still fall within the scope of the claims.

The following additional considerations apply to the foregoing discussion. Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Additionally, certain embodiments are described herein as including logic or a number of routines, subroutines, applications, or instructions. These may constitute either software (e.g., code embodied on a machine-readable medium or in a transmission signal) or hardware. In hardware, the routines, etc., are tangible units capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion) as a hardware module that operates to perform certain operations as described herein.

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

Similarly, the methods or routines described herein may be at least partially processor-implemented. For example, at least some of the operations of a method may be performed by one or more processors or processor-implemented hardware modules. The performance of certain of the operations may be distributed among the one or more processors, not only residing within a single machine, but deployed across a number of machines. In some example embodiments, the processor or processors may be located in a single location, while in other embodiments the processors may be distributed across a number of locations.

The performance of certain of the operations may be distributed among the one or more processors, not only residing within a single machine, but deployed across a number of machines. In some example embodiments, the one or more processors or processor-implemented modules may be located in a single geographic location (e.g., within a home environment, an office environment, or a server farm). In other embodiments, the one or more processors or processor-implemented modules may be distributed across a number of geographic locations.

This detailed description is to be construed as exemplary only and does not describe every possible embodiment, as describing every possible embodiment would be impractical, if not impossible. A person of ordinary skill in the art may implement numerous alternate embodiments, using either current technology or technology developed after the filing date of this application.

Those of ordinary skill in the art will recognize that a wide variety of modifications, alterations, and combinations can be made with respect to the above described embodiments without departing from the scope of the invention, and that such modifications, alterations, and combinations are to be viewed as being within the ambit of the inventive concept.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A digital imaging and artificial intelligence (AI)-based system configured to analyze product dosing, the digital imaging and AI-based system comprising:
one or more processors;
a dosing application (app) comprising computing instructions configured to execute on the one or more processors; and
a dosing learning model, accessible by the dosing app, and trained with dosage data of one or more products, pixel data of a plurality of training images depicting the one or more products, and one or more product appliances or product implements associated with the one or more products, the dosing learning model trained to output analysis of one or more dosages corresponding to the one or more products based on one or more corresponding target dosages applied at different times during a product application lifecycle,
wherein the computing instructions of the dosing app when executed by the one or more processors, cause the one or more processors to:
detect, based on product data, a product identifier of a product,
obtain a set of one or more images of the product, the set of one or more images comprising pixel data as captured by an imaging device, and the pixel data depicting a dosage of the product and at least one of (a) a product appliance configured to apply the product and (b) a product implement configured to receive the product,
generate, based on output of the dosing learning model, a first analysis comprising a dosage comparison comparing the dosage of the product as depicted in pixel data to a target dosage of the product at a first time state, the target dosage of the product defining an expected dosage of the product at the first time state, and
output, based on the dosage comparison, a feedback indication designed to address at least one feature identifiable within the pixel data comprising the dosage of the product.

2. The digital imaging and AI-based system of claim 1, wherein the computing instructions of the dosing app when executed by the one or more processors, further cause the one or more processors to:
obtain a second set of one or more images of the product, the second set of one or more images comprising second pixel data as captured by the imaging device, and the second pixel data depicting a second dosage of the product and the product appliance or the product implement,
generate, based on output of the dosing learning model, a second analysis comprising a second dosage comparison of the second dosage of the product as depicted in second pixel data to a second target dosage of the product at a second time state, the second target dosage of the product defining a second expected dosage of the product at the second time state, and
output, based on the second dosage comparison, a second feedback indication designed to address at least one feature identifiable within the second pixel data comprising the second dosage of the product.

3. The digital imaging and AI-based system of claim 1, further comprising a product-based learning model, accessible by the dosing app, and trained with pixel data of a plurality of training images depicting one or more products, the product-based learning model trained to output product predictions of one or more product identifiers corresponding to the one or more products depicted within the pixel data of the plurality of training images,
and wherein the computing instructions of the dosing app when executed by the one or more processors, further cause the one or more processors to:
obtain a set of one or more images of the product, wherein the product data comprises the set of one or more images of the product, the set of one or more images of the product comprising pixel data of the product as captured by an imaging device, and the pixel data of the product depicting least a portion of the product,
detect, based on output of the product-based learning model inputting the pixel data of the product, the product identifier of the product.

4. The digital imaging and AI-based system of claim 4, wherein the one or more product identifiers are based on one or more features identifiable within the pixel data of the plurality of training images, the one or more features selected from a product category of the one or more products, a product brand of the one or more products, a product variant of the one or more products, a product form of the one or more products, a product packaging of the one or more products and combinations thereof.

5. The digital imaging and AI-based system of claim 4, wherein the output of the product-based learning model comprises a product prediction with a 90% or greater percentage accuracy that the product identifier correctly identifies the product.

6. The digital imaging and AI-based system of claim 1, wherein the dosage data of one or more products comprise at least one of: (a) an amount, size, or dimension of the product; (b) an amount, size, or dimension of the product relative to the product appliance and/or the product implement; and/or (c) a composition of the product.

7. The digital imaging and AI-based system of claim 1, wherein each image of the one or more of first plurality of training images or the first set of one or more images comprises at least one cropped image removing at least a portion of personally identifiable information (PII) of a user.

8. The digital imaging and AI-based system of claim 1, wherein the product identifier is submitted as an input to look up or link to additional data defining the product, the additional data being selected from formula specification of the product, a trait of the product, packaging data of the product, a clinical indication of the product and combinations thereof.

9. The digital imaging and AI-based system of claim 1, wherein each image of the plurality of training images comprises multiple angles or perspectives depicting the one or more products, and wherein each image of the plurality of training images comprises multiple angles or perspectives depicting the one or more dosages.

10. The digital imaging and AI-based system of claim 1, wherein the dosing learning model comprises a segmentation model trained to generate a segmentation mapping defining, in the pixel data, the dosages of the product and the product appliance or the product implement configured to apply the dosages.

11. The digital imaging and AI-based system of claim 1, wherein the feedback indication is generated based on the dosing comparison and at least one of (a) a physical attribute of the product appliance or the product implement; (b) a pattern or arrangement of the product as positioned on or with respect to the product appliance or the product implement; and (c) a provision of the dosage of the product by a user when applying the product with the product appliance or the product implement.

12. The digital imaging and AI-based system of claim 1, wherein the feedback indication comprises at least one of (a) a qualitative rating; (b) a numeric assessment; (c) a visual projection; (d) an augmented reality annotation; (e) and a categorical rating.

13. The digital imaging and AI-based system of claim 1, wherein the target dosage comprises at least one of a visual appearance of the product, a color of the product, a volume of the product, an amount of the product, a dimension of the product, a pattern of the product, a shape of application of the product, a texture of the product, a density of the product, a relative ratio of the product, and/or a position of the product relative to the product appliance or the product implement.

14. The digital imaging and AI-based system of claim 1, wherein the computing instructions of the dosing app when executed by the one or more processors, further cause the one or more processors to render, on a display screen of a computing device, the feedback indication to indicate a difference or similarity between the dosage of the product and the target dosage of the product.

15. The digital imaging and AI-based system of claim 1, wherein the computing instructions of the dosing app when executed by the one or more processors, further cause the one or more processors to render, on a display screen of a computing device, at least one dosage recommendation based on the feedback indication.

16. The digital imaging and AI-based system of claim 1, wherein the one or more processors comprises a server processor of a server, wherein the server is communicatively coupled to a computing device via a computer network, and where the dosing app comprises a server app portion configured to execute on the one or more processors of the server and a computing device app portion configured to execute on one or more processors of the computing device, the server app portion configured to communicate with the computing device app portion, wherein the server app portion is configured to implement one or more of: (1) detecting, based on the product data, the product identifier of the product; (2) obtaining the set of one or more images of the product; (3) generating, based on the output of the dosing learning model, the first analysis comprising a dosage comparison; and/or (4) outputting, based on the dosage comparison, the feedback indication.

17. A digital imaging and artificial intelligence (AI)-based method for analyzing product usage, the digital imaging and AI-based method comprising:
detecting, by one or more processors based on product data, a product identifier of a product,
obtaining, by a dosing application (app) executing on the one or more processors, a set of one or more images of the product, the set of one or more images comprising pixel data as captured by an imaging device, and the pixel data depicting a dosage of the product and at least one of: (a) a product appliance configured to apply the product, or (b) a product implement configured to receive the product,
generating, based on output of a dosing learning model, a first analysis comprising a dosage comparison comparing the dosage of the product as depicted in pixel data to a target dosage of the product at a first time state, the target dosage of the product defining an expected dosage of the product at the first time state, wherein the dosing learning model executes on the one or more processors and is trained with dosage data of one or more products that includes the product, pixel data of a plurality of training images depicting the one or more products, and one or more product appliances or product implements associated with the one or more products, the dosing learning model trained to output analysis of one or more dosages corresponding to the one or more products based on one or more corresponding target dosages applied at different times during a product application lifecycle, and
outputting, by the one or more processors based on the dosage comparison, a feedback indication designed to address at least one feature identifiable within the pixel data comprising the dosage of the product.

18. The digital imaging and AI-based method of claim 33 further comprising:
obtaining a second set of one or more images of the product, the second set of one or more images comprising second pixel data as captured by the imaging device, and the second pixel data depicting a second dosage of the product and the product appliance or the product implement,
generating, based on output of the dosing learning model, a second analysis comprising a second dosage comparison of the second dosage of the product as depicted in second pixel data to a second target dosage of the product at a second time state, the second target dosage of the product defining a second expected dosage of the product at the second time state, and
outputting, based on the second dosage comparison, a second feedback indication designed to address at least one feature identifiable within the second pixel data comprising the second dosage of the product.

19. The digital imaging and AI-based method of claim 33, further comprising a product-based learning model, accessible by the dosing app, and trained with pixel data of a plurality of training images depicting one or more products, the product-based learning model trained to output product predictions of one or more product identifiers corresponding to the one or more products depicted within the pixel data of the plurality of training images,
and wherein the AI-based method further comprises:
obtaining a set of one or more images of the product, wherein the product data comprises the set of one or more images of the product, the set of one or more images of the product comprising pixel data of the product as captured by an imaging device, and the pixel data of the product depicting least a portion of the product,
detecting, based on output of the product-based learning model inputting the pixel data of the product, the product identifier of the product.

20. A tangible, non-transitory computer-readable medium storing instructions for analyzing product usage, that when executed by one or more processors cause the one or more processors to:
detect, based on product data, a product identifier of a product,
obtain, by a dosing application (app), a set of one or more images of the product, the set of one or more images comprising pixel data as captured by an imaging device, and the pixel data depicting a dosage of the product and at least one of: (a) a product appliance configured to apply the product, or (b) a product implement configured to receive the product,
generate, based on output of a dosing learning model, a first analysis comprising a dosage comparison comparing the dosage of the product as depicted in pixel data to a target dosage of the product at a first time state, the target dosage of the product defining an expected dosage of the product at the first time state, wherein the dosing learning is trained with dosage data of one or more products that includes the product, pixel data of a plurality of training images depicting the one or more products, and one or more product appliances or product implements associated with the one or more products, the dosing learning model trained to output analysis of one or more dosages corresponding to the one or more products based on one or more corresponding target dosages applied at different times during a product application lifecycle, and
output, based on the dosage comparison, a feedback indication designed to address at least one feature identifiable within the pixel data comprising the dosage of the product.
